(19) United States
(12) Patent Application Publication
(10) Pub. No.: US 2024/0201031 A1
(43) Pub. Date: Jun. 20, 2024

(54) MODULAR WEARABLE INTERFACE DEVICES

(71) Applicant: Cornell University, Ithaca, NY (US)

(72) Inventors: Hsin-Liu Kao, Ithaca, NY (US); Pin-Sung Ku, Ithaca, NY (US)

(21) Appl. No.: 18/573,990

(22) PCT Filed: Jun. 22, 2022
(86) PCT No.: PCT/US2022/034490

§ 371 Date: Dec. 22, 2023

Related U.S. Application Data
(60) Provisional application No. 63/213,647, filed on Jun. 22, 2021.

(51) Int. Cl. G01L 1/14; G06F 1/16; G06F 3/01; H05K 1/02; H05K 1/18

(52) U.S. Cl. CPC G01L 1/142; G06F 1/163; G06F 3/016; H05K 1/0283; H05K 1/189

(57) ABSTRACT

This technology is directed to modular wearable devices comprising interchangeable functional modules. The modular wearable device is re-configurable, reusable, and extensible comprising skin-conformable base substrates, reusable functional modules, and reusable wire modules. The functional modules are attachable and removable in a plug-and-play type construction. The functional modules may be preprogrammed and connected in unique sequences to achieve customized functions. The functional modules include power, sensor, modifier, and actuator modules that are interchangeable to customize the functionality of the modular wearable device. The functional modules are affixed to a wire module assembly, the wire module assembly providing the infrastructure for power and communications. The sensor module receives an input signal and transmits an output signal to the actuator module to initiate a response function. In certain embodiments, a modifier module may alter the output signal and transmit the altered output signal to the actuator

### CROSS-REFERENCE TO RELATED APPLICATIONS

This application claims the benefit of and priority to U.S. Provisional Patent Application No. 63/213,647, filed Jun. 22, 2021 and titled “Woven On-Skin Electronic Devices And Methods Of Fabricating The Same,” the entire contents of which are hereby fully incorporated herein by reference.

### TECHNICAL FIELD

This disclosure relates to modular wearable on-skin interface devices with tactile functionality.

### BACKGROUND

Electronic devices have undergone dramatic transformations over the last several decades. As electronic devices are increasingly being used throughout the day for various tasks, new form factors are being developed. Form factors have been developed that use the human skin as an interface to facilitate human-computer interactions (“HCl”). Developing a new fabrication process for conventional on-skin interface devices is time consuming, challenging to incorporate new features, and does not allow for quick form factor preview through prototyping. Conventional on-skin interface devices include long fabrication times, a fixed circuit layout after fabrication, and do not support form-factor preview on different body locations. Conventional fabrication methods, including laser patterning, inkjet printing, ink deposition, lamination, embedding, molding, and casting, do not support customized functionality of the on-skin interface device.

Conventional on-skin interface devices are typically body-mounted devices, e.g., watches, accessories, pod-like devices, or garments. Body-mounted devices protrude from the body limiting wearability, require users to remember to wear the device daily, and confine the technology to one body location. Garments may lack precise fit for effective placement and may not be configured for long term use, i.e., the garments may not be substantially waterproof for launderability.

Conventional on-skin interfaces may include tactile interfaces. While tactile interfaces have utilized skin as an area for haptic input, bulky form factors and complicated mechanical systems have hindered wider utilization of body locations. Form factors in such interfaces are typically contained to wristbands, limiting application to only the forearm. Conventional methods for high-resolution tactile outputs are often bulky and not body conformable. Conventional methods often require rigid devices, which may not be wearable and can constrain the use of conventional on-skin interfaces to certain body locations.

### BRIEF DESCRIPTION OF THE DRAWINGS

FIG. 1 is a block diagram depicting a modular wearable device system.

FIG. 2A is a block diagram depicting a wire module assembly, and FIG. 2B is a block diagram depicting a wire module.

FIG. 3A is a block diagram depicting a power module, FIG. 3B is a block diagram depicting a sensor module, FIG. 3C is a block diagram depicting a modifier module, and FIG. 3D is a block diagram depicting an actuator module.

FIG. 4A depicts an exploded view of a substrate, FIGS. 4B-4C depict an asymmetric configuration of sections of the substrate, and FIGS. 4D-4E depict a symmetric configuration of sections of the substrate.

FIGS. 5A-5D depict example embodiments of a modular wearable device.

FIG. 6A depicts an example embodiment of a wrist mounted modular wearable device and FIG. 6B depicts an enlargement of a sensor module of the wrist mounted modular wearable device.

FIGS. 7A-7D depict example embodiments to connect a functional module to a single wire module, and FIGS. 7E-7F depict example embodiments to connect a functional module between two wire modules.

FIGS. 8A-8C depict example functional module connection configurations, FIGS. 8D-8I depict example substrate and wire module configurations, and FIG. 8J depicts an example circuit layout of a wire module assembly.

FIG. 9 is a block diagram depicting a computing machine and a module.

### DETAILED DESCRIPTION

### Overview

The present technology is directed to modular, wearable, on-skin interface devices with tactile functionality. The modular wearable device is a construction toolkit for on-skin interface devices that is re-configurable, reusable, and extensible. The modular wearable device has 1) a form factor for attaching to a user's skin, 2) robust yet slim connection mechanisms between modules, 3) reconfigurability of circuit components for extensible prototyping, and 4) fast and flexible device prototyping that allows iterative design processing and quick on-body preview. The modular wearable device comprises skin-conformable base substrates, reusable functional modules, and reusable wire modules. A set of wire modules may be interconnected to create a customized form factor, or wire module assembly, for the modular wearable device. The set of wire modules when interconnected form a circuit to which the functional modules may be attached. The functional modules are attachable and removable from the wire module assembly in a plug-and-play type construction. The functional modules may be preprogrammed and connected in unique sequences to achieve various customized functions. The functional modules include power modules, sensor modules, modifier modules, and actuator modules. A user can select a particular type of each functional module and affix the functional modules to a wire module assembly. The functional modules are interchangeable to customize the functionality of the modular wearable device. The modular wearable device includes single function modules made of pre-programmed slim, flexible printed circuit board; slim, flexible, and skin-conformable substrate pieces that serve as the infrastructure connecting the functional modules; and a base that conforms and adheres to human skin. The functional modules are reusable, reconfigurable, and easily attach/detach, enabling extensible circuit function customization. The flexible wire modules overcome challenging body locations and provide stable power transmission and signal communication between the functional modules.

Each functional module works contingently with a received signal and generates an output signal for the next module. The power module provides power, which is transmitted to the other functional modules via the wire modules. The wire modules, when connected as a wire module assembly, serve as the power and communication infrastructure for the functional modules. The sensor module receives an input and transmits an output to the actuator module to initiate a response function. Alternately, the sensor module receives an input and transmits an output to the modifier module, which transforms the signal and transmits the transformed signal to the actuator module to initiate an altered response function. Response functions include, but are not limited to, a force, vibration, thermal sensation (heat), motion, variable stiffness, thermochromic display, audio, light (light emitting diode (“LED”) or organic light emitting diode (“OLED”)), or photochromic display.

The modular wearable device may be configured to be affixed to a user or a user's garment by an adhesive layer. In an alternate embodiment, the modular wearable device may have fasteners, clips, or other suitable mechanisms to be affixable to the user.

The modular wearable device is customizable for various types of skin topographies. The modular wearable device can be customized to be affixed to an underlying skin topography or body landmark. The modular wearable device can be customized for placement on planar body parts (e.g., back of hand), cylindrical body parts (e.g., forearm), protruded body joints (e.g., elbow, knees, and knuckles), and concave body locations (e.g., the purlicue, armpit, and Achilles tendon arch). The ability to customize the modular wearable device in size, shape, and for diverse use on diverse body locations eliminates the need for prototype devices.

These and other aspects, objects, features, and advantages of the disclosed technology will become apparent to those having ordinary skill in the art upon consideration of the following detailed description of illustrated examples.

### Example System Architecture

Turning now to the drawings, in which like numerals indicate like (but not necessarily identical) elements throughout the figures, examples of the technology are described in detail.

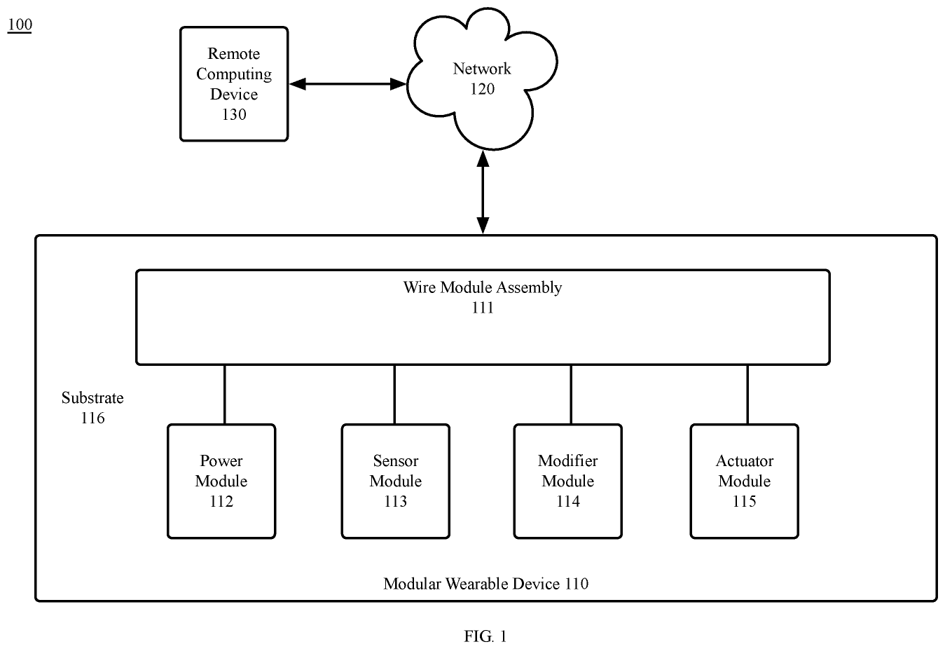

FIG. 1 is a block diagram depicting a modular wearable device system 100, in accordance with certain examples. As depicted in FIG. 1, the modular wearable device system 100 comprises a modular wearable device 110 and a remote computing device 130. Modular wearable device 110 and remote computing device are configured to communicate via a network 120.

In example embodiments, network 120 includes one or more wired or wireless telecommunications systems by which network devices may exchange data. For example, the network 120 may include one or more of a local area network (“LAN”), a wide area network (“WAN”), an intranet, an Internet, a storage area network (“SAN”), a personal area network (“PAN”), a metropolitan area network (“MAN”), a wireless local area network (“WLAN”), a virtual private network (“VPN”), a cellular or other mobile communication network, a BLUETOOTH® wireless technology connection, a near field communication (“NFC”) connection, any combination thereof, and any other appropriate architecture or system that facilitates the communication of signals, data, and/or messages.

Modular wearable device 110 is a construction toolkit for on-skin interface devices that is reconfigurable, reusable, and extensible. Modular wearable device 110 comprises skin-conformable base substrates, reusable functional modules, and reusable wire modules. The functional modules are attachable and removable in a plug-and-play type construction. The functional modules may be preprogrammed and connected in unique sequences to achieve various customized functions. The functional modules comprise a flexible substrate. The flexible substrate comprises at least one flexible, i.e., stretchable and/or bendable, layer or a plurality of flexible layers including at least one adhesive layer and at least one supporting layer, such as substrate 116 described in greater detail herein. Each functional module comprises 1) at least two electrodes, such that a voltage can be applied between the at least two electrodes and optionally at least one ground electrode, 2) at least one input connector, 3) at least one output connector, 4) at least one functional flexible printed circuit board (“FPCB”), 5) optionally one or more programming pins, and 6) at least one microcontroller unit. The functional modules include power modules, sensor modules, modifier modules, and actuator modules. A user can select a particular type of each functional module and affix the functional modules to a wire module assembly in a plug-and-play type construction. The functional modules are interchangeable to customize the functionality of modular wearable device 110. Modular wearable device 110 includes 1) single-function (functional) modules made of pre-programmed slim, FPCBs, and 2) slim, flexible, and skin-conformable substrate pieces that serve as the infrastructure connecting the functional modules, and a base that conforms and adheres to human skin. The functional modules are reusable, reconfigurable, and easy to attach/detach, enabling extensible circuit function customization. In an alternate embodiment, one or more self-assembly protoboards may be used in place of the single function modules. Each self-assembly protoboard comprises a plurality of connectors to affix a plurality of different types of modules. The flexible wire modules overcome challenging body locations and provide stable power transmission and signal communication between the functional modules.

Each functional module works contingently with a received signal and generates an output signal for the next module. The functional modules include a power module, a sensor module, an actuator module, and, optionally, a modifier module. The power module provides power, which is transmitted to the other functional modules via the wire modules, which serve as the power and communication infrastructure for the functional modules. The sensor module receives an input and transmits an output to the actuator module to initiate a response function. Alternately, the sensor module receives an input and transmits an output to the modifier module, which transforms the signal and transmits the transformed signal to the actuator module to initiate an altered response function. Response functions include, but are not limited to, a force, vibration, thermal sensation (heat), motion, variable stiffness, thermochromic display, audio, light (light emitting diode (“LED”) or organic light emitting diode (“OLED”)), or photochromic display.

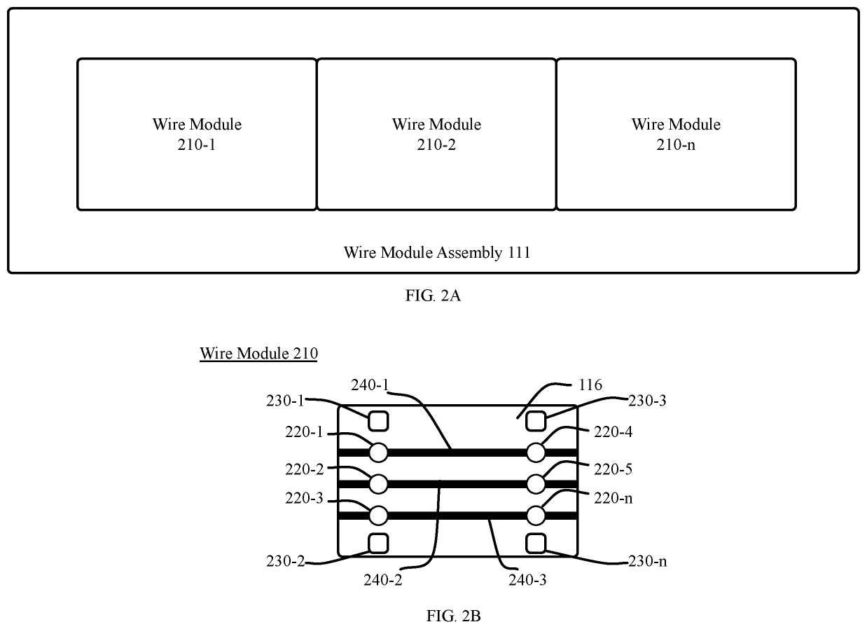

Modular wearable device 110 comprises a wire module assembly 111, a power module 112, a sensor module 113, a modifier module 114 (optional), an actuator module 115, and a substrate 116. Power module 112, sensor module 113, modifier module 114, and actuator module 115 may be collectively referred to herein as functional modules. In an alternate embodiment, modular wearable device does not comprise a modifier module 114. The wire module assembly 111 is described in greater detail herein with reference to FIG. 2A.

FIG. 2A is a block diagram depicting a wire module assembly 111, in accordance with certain examples. Wire module assembly 111 is comprised of a plurality of wire modules 210. As depicted in FIG. 2A, wire module assembly 111 is comprised of wire modules 210-1, 210-2, through 210-n, where “n” represents a quantity of wire modules 210 configured for a particular form factor of modular wearable device 110. Each wire module 210 is coupled to an adjacent wire module 210 such that electrical conductivity is maintained across the wire module assembly 111. While FIG. 2A depicts wire modules 210 connected in a linear fashion, any configuration of wire modules 210 may be used to 1) form a circuit for modular wearable device 110, and 2) customize a form factor of modular wearable device 110 by customizing the configuration of the wire module assembly 111 as desired for a particular application of modular wearable device 110. For example, wire modules 210 may be connected end to end, at a 900 angle relative to a next wire module 210, in a stacked configuration, or any other suitable orientation. Wire modules 210 are described in greater detail herein with reference to FIG. 2B.

FIG. 2B is a block diagram depicting a wire module 210, in accordance with certain examples. Wire modules 210 provide the power and communication infrastructure for the functional modules, such as power module 112, sensor module 113, modifier module 114, or actuator module 115. As depicted in FIG. 2B, wire module 210 comprises substrate 116, wire module couplers 220, functional module couplers 230, and conductors 240. Substrate 116 is described in greater detail herein with reference to FIG. 1.

Wire module couplers 220 are coupling devices configured to affix a wire module 210 to one or more additional wire modules 210 while maintaining conductivity between conductors 240 of the affixed wire modules 210 such that a circuit is formed. Wire module couplers 220 may be configured such that wire module couplers 220 on a first wire module 210 mate with wire module couplers 220 on a second wire module 210. Wire module couplers 220 may be located on either atop or bottom surface of substrate 116 of wire modules 210. In an example, wire module couplers 220-1, 220-2, and 220-3 may be located on a top surface of substrate 116 of wire module 210-1 and may mate with wire module couplers 220-4, 220-5, and 220-6 located on a bottom surface of the substrate 116 of wire module 210-2. In an alternate example, wire module couplers 220-1, 220-2, and 220-3 may be located on a top surface of substrate 116 of wire module 210-1 and may mate with wire module couplers 220-4, 220-5, and 220-6 also located on a top surface of the substrate 116 of wire module 210-2. Wire module couplers 220 may be any suitable coupling device, or combination of coupling devices, including, but not limited to, snaps, hook and loops, magnetic pairs, z-axis conductive tape, and/or pin/hole assemblies. While six wire module couplers 220 are depicted in FIG. 2B, any suitable quantity of wire module couplers 220 may be used to affix a wire module 210 to one or more additional wire modules 210 while maintaining conductivity between conductors 240 of the affixed wire modules 210. For example, one, two, three, four, five, or more wire module couplers 220 may be used. While six wire module couplers 220 are depicted in two vertical, linear configurations in FIG. 2B, any suitable arrangement of wire module couplers 220 may be used to affix a wire module 210 to one or more additional wire modules 210 while maintaining conductivity between conductors 240 of the affixed wire modules 210. In an example, the wire module couplers 220 may be aligned in two sloping linear configurations. In an alternate example, the wire modular couplers 220 may in a non-linear configuration while still being a suitable arrangement to affix a wire module 210 to one or more additional wire modules 210 while maintaining conductivity between conductors 240 of the affixed wire modules 210.

Functional module couplers 230 are coupling devices that affix a functional module, such as power module 112, sensor module 113, modifier module 114, or actuator module 115, to one or more wire modules 210 such that the functional modules are conductively connected to the conductors 240 that form the circuit of wire module assembly 111. Functional module couplers 230 are configured such that functional module couplers 230 on a functional module mate with functional module couplers 230 on one or more wire modules 210. Functional module couplers 230 may be any suitable coupling device including, but not limited to, snaps, hook and loops, magnetic pairs, z-axis conductive tape, or pin/hole assembly. In an example, functional module couplers 230 may be a same type of coupling device as wire module couplers 220. For example, functional module couplers 230 and wire module couplers 220 may both be magnetic pairs. In an alternate example, functional module couplers 230 may not be the same type of coupling devices as wire module couplers 220. While four functional module couplers 230 are depicted in FIG. 2B, any suitable quantity of functional module couplers 230 may be used to affix a functional module to one or more wire modules 210 while maintaining a conductive connection between conductors 240 of the affixed wire modules 210. For example, one, two, three, five, six, or more functional module couplers 230 may be used. While four functional module couplers 230 are depicted in two vertical, linear configurations in FIG. 2B, any suitable arrangement of functional module couplers 230 may be used to affix a functional module to one or more wire modules 210 while maintaining a conductive connection between the functional module and conductors 240 of the affixed wire modules 210. In an example, the functional module couplers 230 may be aligned in two sloping linear configurations. In an alternate example, functional modular couplers 230 may in a non-linear configuration while still being a suitable arrangement to affix a functional module to one or more wire modules 210 while maintaining a conductive connection between the functional module and conductors 240 of the affixed wire modules 210.

Wire module 210 comprises conductors 240. In an example, conductors 240 are conductive traces that connect the functional modules. Conductors 240 provide voltage (power), ground, and signal connections for the functional modules. In an example, conductor 240-1 provides voltage to the functional modules, conductor 240-2 provides a signal connection between the functional modules, and conductor 240-3 provides a ground to the functional modules. In an example, conductors 240 are comprised of conductive fabric tape affixed to substrate 116. In an example, conductors 240 are insulated copper wires that are affixed to substrate 116 in a serpentine pattern such that wire module 210 is a stretchable module. Conductors 240 may be any suitable flexible, conductive material.

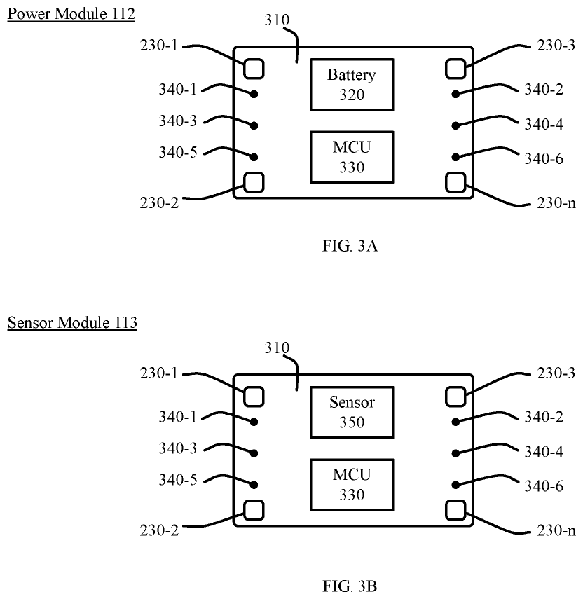

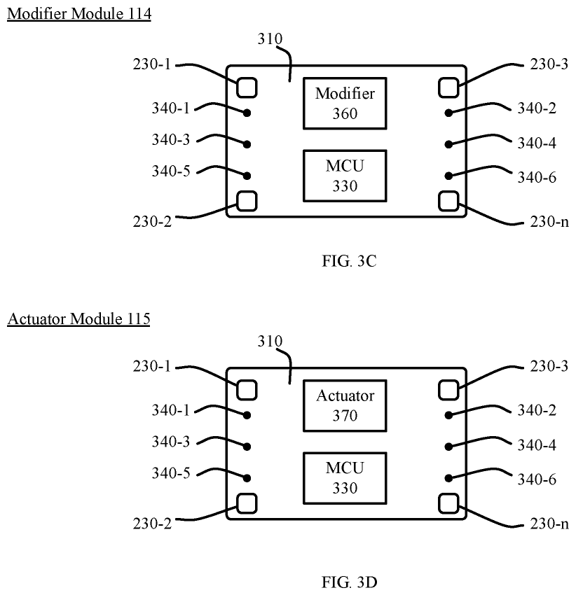

Returning to FIG. 1, modular wearable device 110 comprises a power module 112. Power module 112 is described in greater detail herein with reference to FIG. 3A.

FIG. 3A is a block diagram depicting a power module 112, in accordance with certain examples. Power module 112 comprises functional module couplers 230, flexible printed circuit board (“FPCB”) 310, battery 320, microcontroller unit (“MCU”) 330, and circuit connectors 340. Functional module couplers 230 were previously described herein with reference to FIG. 2B. FPCB 310, which also may be referred to as a flex print or a flex circuit, is a circuit board that can be bent into a desired shape. For example, FPCB 310 may bend to conform to a location on a user of modular wearable device 110. FPCB 310 may conform to locations such as a knee, an elbow, or a wrist. In an example, FPCB 310 may bend in response to a motion of a user, such as the movement of a knee, an elbow, or a wrist. FPCB 310 may be a single sided circuit board, a single sided circuit board with dual access, a double-sided circuit board, a multi-layer circuit board, or any other suitable type of circuit board. FPCB 310 may be any suitable shape including, but not limited to, square, rectangular, hexagonal, octagonal, or circular. The width and length range of FPCB 310 may be 10 mm to 50 mm, with a preferred width and length range of 20 mm to 40 mm, with an optimal width and length range of 20 mm to 30 mm. The thickness range of FPCB 310, excluding affixed components, may be 0.1 mm to 2 mm, with a preferred thickness range of 0.1 mm to 1 mm, with an optimal thickness range of 0.1 mm to 0.5 mm.

Power module 112 comprises battery 320. In an example, battery 320 may be a lithium polymer (“LiPo”) battery, a lithium ceramic battery, a triboelectric nanogenerator (“TENG”), or any other suitable slim form factor power source for power module 112. Battery 320 is affixed to FPCB 310 such that the terminals of battery 320 are conductively connected to the conductor 240 providing voltage to the functional modules of one or more wire modules 210 via circuit connectors 340.

Power module 112 comprises MCU 330. MCU 330 may be a general purpose processor, a processor core, a reconfigurable processor, a printed circuit board (“PCB”), a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. MCU 330 may be powered by battery 320.

In an example, MCU 330 is a pre-programmed MCU. In an alternate example, MCU 330 is programmable via programming pins on the functional modules (not depicted). In an example, MCU 330 is an ATtiny85 MCU.

In an example, MCU 330 has a small form factor and is affixed to FPCB 310 such that MCU 330 receives voltage (power) from FPCB 310. In an example, MCU 330 may comprise Inter-Integrated Circuit (“I2C”) interfaces, serial peripheral interfaces (“SPI”), or Universal Asynchronous Receiver/Transmitter (“UART”) interfaces to interface with other functional modules. In an example, MCU 330 may be configured to communicate with external computing systems or other computing devices by a radio frequency identification (“RFID”) signal, for example, remote computing device 130. In an example, MCU 330 may be configured to communicate with external computing systems or other computing devices via Bluetooth, for example, remote computing device 130.

Power module 112 comprises circuit connectors 340. In an example, circuit connectors 340 are metal or conductive pads that are located on the bottom surface of the functional modules. Circuit connectors 340 are configured such that circuit connectors 340 align with conductors 240-1, 240-2, and 240-3. For example, circuit connectors 340-1 and 340-2 are configured to align with conductor 240-1 such that the functional module, i.e., power module 112, sensor module 113, modifier module 114, or actuator module 115, is conductively connected to the conductor 240-1 of the wire module 210. Circuit connectors 340-3 and 340-4 are configured to align with conductor 240-2 and circuit connectors 340-5 and 340-6 are configured to align with conductor 240-3.

Returning to FIG. 1, modular wearable device 110 comprises a sensor module 113. Sensor module 113 is described in greater detail herein with reference to FIG. 3B.

FIG. 3B is a block diagram depicting a sensor module 113, in accordance with certain examples. Sensor module 113 is configured to receive an input and transmit an output signal to generate a response function. Sensor module 113 transmits the output signal via conductor 240-2 to modifier module 114 or actuator module 115. Sensor module 113 comprises functional module couplers 230, FPCB 310, MCU 330, circuit connectors 340, and sensor 350. Functional module couplers 230 were previously described herein with reference to FIG. 2B. FPCB 310, MCU 330, and circuit connectors 340 were previously described herein with reference to FIG. 3A.

Sensor module 113 comprises a sensor 350. Sensor 350 is a component that detects one or more inputs from the environment in which modular wearable device 110 is located. In an example, sensor 350 has a small form factor such that sensor 350 may be affixed to sensor module 113 without adding a significant amount of thickness to FPCB 310. In an example, sensor 350 is affixed to sensor module 113 without extending beyond the outer dimensions of the FPCB 310. In an alternate example, sensor 350 may comprise a sensor component that is extended from FPCB 310 such that the extendable sensor component may be in contact with a location of the user outside of the form factor of the modular wearable device 110. Extending the sensor component allows for repositioning of the sensor at a different body location away without repositioning the modular wearable device 110. Sensor 350 may be one or more of a capacitive touch sensor, a resistive sensor, a strain sensor, a pressure sensor, a biosensor, an ultraviolet (“UV”) light sensor, an environmental gas sensor, an inertial movement unit (“IMU”), a microphone, a water sensor, a velocity sensor, a physiological sensor, or any other suitable sensor to detect an input from the environment in which modular wearable device 110 is located. In an example, a biosensor may be configured to monitor or measure one or more of a temperature, blood pressure, pulse, or any other suitable biometric.

Returning to FIG. 1, modular wearable device 110 comprises a modifier module 114. Modifier module 114 is described in greater detail herein with reference to FIG. 3C.

FIG. 3C is a block diagram depicting a modifier module 114, in accordance with certain examples. Modifier module 114 is a module configured to transform input signals and transmit output signals. In an example, modifier module 114 is configured to receive a signal from sensor module 113, alter the signal, and transmit the altered signal via conductor 240-2 to actuator module 115. Modifier module 114 comprises functional module couplers 230, flexible printed circuit board 310, microcontroller unit 330, circuit connectors 340, and modifier 360. Functional module couplers 230 were previously described herein with reference to FIG. 2B. Flexible printed circuit board 310, microcontroller unit 330, and circuit connectors 340 were previously described herein with reference to FIG. 3A.

Modifier module 114 comprises modifier 360. In an example, modifier 360 has a small form factor such that modifier 360 may be affixed to modifier module 114 without adding a significant amount of thickness to FPCB 310. Modifier 360 is a device that alters a feature of the response function of actuator module 115. For example, modifier 360 may alter the amplitude of the response function of actuator module 115. In an example, the amplitude may be associated with response functions including light, volume, vibration, and heat.

Returning to FIG. 1, modular wearable device 110 comprises an actuator module 115. Actuator module 115 is described in greater detail herein with reference to FIG. 3D.

FIG. 3D is a block diagram depicting an actuator module 115, in accordance with certain examples. Actuator module 115 functions to initiate a response function based on a signal received from sensor module 113 or modifier module 114. Response functions include, but are not limited to, a force, vibration, thermal sensation (heat), motion, variable stiffness, thermochromic display, audio, light (LED or OLED), or photochromic display. Actuator module 115 comprises functional module couplers 230, flexible printed circuit board 310, microcontroller unit 330, circuit connectors 340, and actuator 370. Functional module couplers 230 were previously described herein with reference to FIG. 2B. Flexible printed circuit board 310, microcontroller unit 330, and circuit connectors 340 were previously described herein with reference to FIG. 3A.

Actuator module 115 comprises actuator 370. Actuator 370 may also be referred to as a functional device. Actuator 370 is a component configured to provide a response function based on one or more signals or inputs from sensor module 113 or modifier module 114. In an example, actuator 370 is affixed to actuator module 115 without extending beyond the outer dimensions of the FPCB 310. In an alternate example, actuator 370 may comprise an actuator component that is extended from FPCB 310 such that the extendable actuator component may be in contact with a location of the user outside of the form factor of the modular wearable device 110. Extending the actuator component allows for repositioning of the actuator 370 at a different body location away without repositioning the modular wearable device 110. In an example, actuator 370 has a small form factor such that actuator 370 may be affixed to actuator module 115 without adding a significant amount of thickness to FPCB 310. As used herein, an actuator 370 comprises a device configured to change from a first state to a second state responsive to a first input. In some aspects, the actuator is further configured to change from the second state back to the first state responsive to a second input, which could be the same input as the first input or a different input than the first input. In some aspects, the actuator is configured to change from a first state to a particular one of a plurality of available states responsive to an input corresponding to that particular one of a plurality of available states. In some examples, the actuator is configured to cycle between a first state and a second state responsive to one or more inputs. In some examples, the actuator is biased toward a first state so that, following actuation of the actuator to change state from the first state to the second state, the actuator will automatically return to the first state under action of the bias. In some examples, actuator 370, in accord with at least some aspects of the present concepts, may include one or more of a haptic feedback component, a stiffness component, a thermochromic display, a photochromic display, an illumination device (such as a light emitting diode (“LED”), an LED array, or organic light emitting diode (“OLED”)), an audio device, a shape-memory alloy (“SMA”) device, an optical fiber, a buzzer or alarm, or any other suitable functional device. The response function of actuator 370 may comprise one or more of a force, a vibration, a motion, a variable-stiffness response, a color change, a light emittance, a thermal sensation, a skin-shifting actuation, a self-shifting actuation, a bending movement, an expanding movement, a shrinking movement, a deformation movement, a pinching movement, a brushing movement, a twisting movement, a lengthening movement, or any other suitable response function. In an example, the thermal sensation may be a warming sensation or a cooling sensation. For example, the haptic feedback component may be a SMA actuator configured to apply a force, a vibration, or a motion. The stiffness component may comprise a SMA actuator to enable variable-stiffness. The thermochromic display may comprise thermochromic materials configured for color change. The SMA device may comprise SMA micro-springs configured to function as skin-shifting actuators when attached to a skin location of a user or as a self-shifting actuator when in close contact to a skin location of a user. The SMA micro-springs may be configured to apply one or more of a compression, a pinch, a brush, or a twist.

In an example, power module 112, sensor module 113, modifier module 114, and actuator module 115 may comprise an outer silicon layer such that each of the modules are waterproof In an example, power module 112, sensor module 113, modifier module 114, and actuator module 115 may be color coded to distinguish each type of module. For example, power module 112 may comprise an MCU 330 that is black, sensor module 113 may comprise an MCU 330 that is yellow, modifier module 114 may comprise an MCU 330 that is blue, and actuator module 115 may comprise an MCU 330 that is white. Any suitable colors may be used to distinguish the modules.

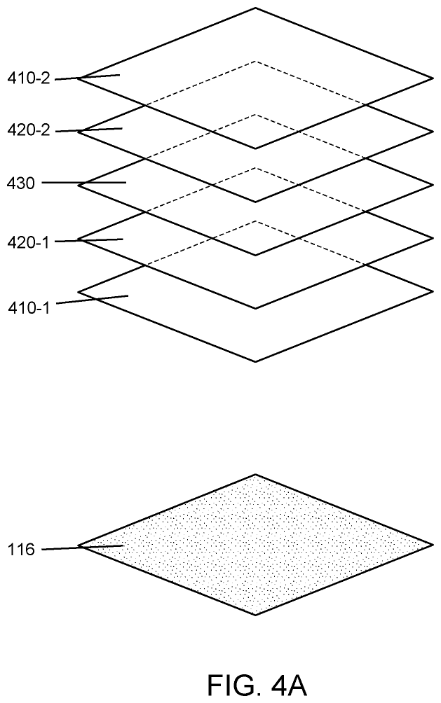

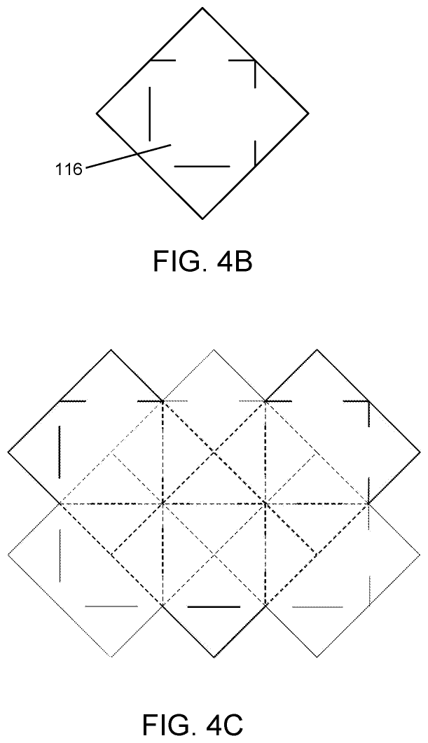

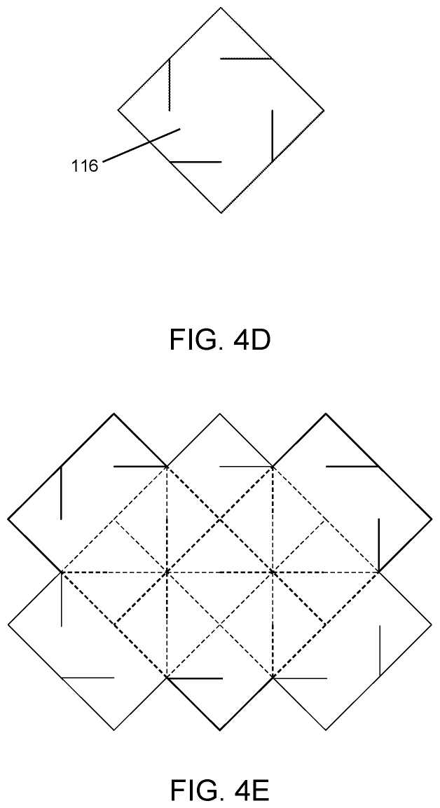

Returning to FIG. 1, modular wearable device 110 comprises a substrate 116. Substrate 116 is described in greater detail herein with reference to FIGS. 4A-4E.

FIG. 4A is an exploded view of substrate 116, in accordance with certain examples. Substrate 116 is a multi-layer slim conformable substrate that serves as a base for the functional modules and the wire modules 210. Substrate 116 comprises silicone layers 410, adhesive layers 420, and stabilizer layer 430. Silicone layer 410-1 is affixed to stabilizer layer 430 by adhesive layer 420-1. In an example, adhesive layers 420 are double sided adhesive layers suitable to affix layers of substrate 116. Stabilizer layer 430 is affixed to silicone layer 410-2 by adhesive layer 420-2. In an example, stabilizer layer 430 may be a polyvinyl alcohol (“PVA”) layer, a silicone layer, a rubber layer, or any other suitable conformable material to provide structure to substrate 116. Substrate 116 may be partitioned into suitable shapes and dimensions for use in constructing modular wearable device 110. In an example, substrate 116 may be partitioned into squares, rectangles, hexagons, octagons, circles, or any other suitable shape.

The width and length range of substrate 116 may be 10 mm to 80 mm, with a preferred width and length range of 20 mm to 60 mm, with an optimal width and length range of 20 mm to 40 mm. The thickness range of substrate 116 may be 10 μm to 1000 μm, with a preferred thickness range of 10 μm to 100 μm, with an optimal thickness range of 10 μm to 40 μm.

FIGS. 4B-4C depict an asymmetric configuration of sections of the substrate 116. In FIG. 4B, asymmetric markings on substrate 116 illustrate cutting lines that can be used to tessellate multiple sections of substrate 116 into a panel as illustrated in FIG. 4C.

FIGS. 4D-4E depict a symmetric configuration of sections of the substrate 116. In FIG. 4D, symmetric markings on substrate 116 illustrate cutting lines that can be used to tessellate multiple sections of substrate 116 into a panel as illustrated in FIG. 4E.

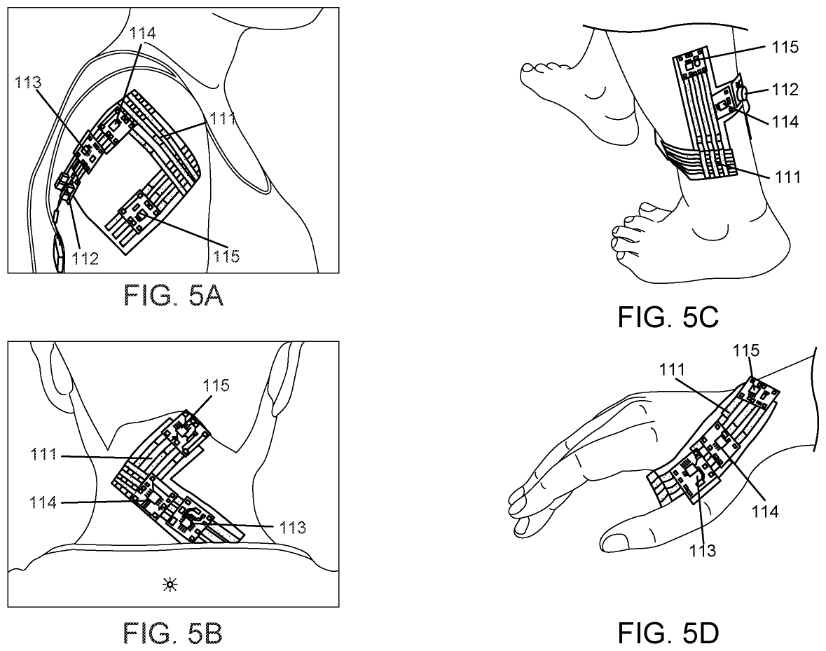

FIGS. 5A-5D depict example embodiments of a modular wearable device 110, in accordance with certain examples. Each of the example embodiments depicted in FIGS. 5A-5D comprise a different configuration for wire module assembly 111 and affixed functional modules. FIG. 5A depicts a shoulder mounted modular wearable device 110. The shoulder mounted modular wearable device 110 is conformably affixed to a user's shoulder. The shoulder mounted modular wearable device 110 comprises a wire module assembly 111 with a power module 112, a sensor module 113, a modifier module 114, and an actuator module 115 affixed to a top surface of the wire module assembly 111.

FIG. 5B depicts a neck mounted modular wearable device 110. The neck mounted modular wearable device 110 is conformably affixed to a user's neck. The neck mounted modular wearable device 110 comprises a wire module assembly 11 Iwith a sensor module 113, a modifier module 114, and an actuator module 115 affixed to a top surface of the wire module assembly 111. A power module 112 is not depicted in FIG. 5B.

FIG. 5C depicts an ankle mounted modular wearable device 110. The ankle mounted modular wearable device 110 is conformably affixed to a user's ankle. The ankle mounted modular wearable device 110 comprises a wire module assembly 111 with a power module 112, a modifier module 114, and an actuator module 115 affixed to a top surface of the wire module assembly 111. A sensor module 113 is not depicted in FIG. 5C.

FIG. 5D depicts a hand mounted modular wearable device 110. The hand mounted modular wearable device 110 is conformably affixed to a user's hand. The hand mounted modular wearable device 110 comprises a wire module assembly 111 with a sensor module 113, a modifier module 114, and an actuator module 115 affixed to a top surface of the wire module assembly 111. A power module 112 is not depicted in FIG. 5B.

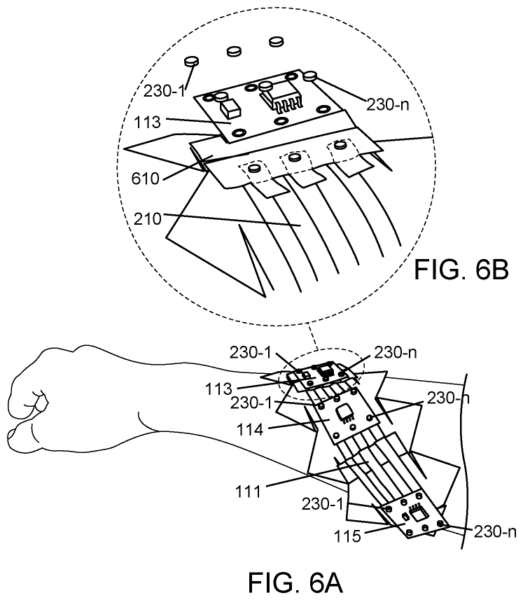

FIG. 6A depicts an example embodiment of a wrist mounted modular wearable device 110 and FIG. 6B depicts an enlargement of the sensor module 113 of the wrist mounted modular wearable device 110, in accordance with certain examples. As depicted in FIG. 6A, the wrist mounted modular wearable device 110 comprises a wire module assembly 111, a sensor module 113, a modifier module 114, an actuator module 115, and functional module couplers 230-1 through 230-n for each of the functional modules (a plurality of functional couplers 230). The sensor module 113, modifier module 114, and actuator module 115 are depicted as being affixed to the wire module assembly 111 by the plurality functional module couplers 230.

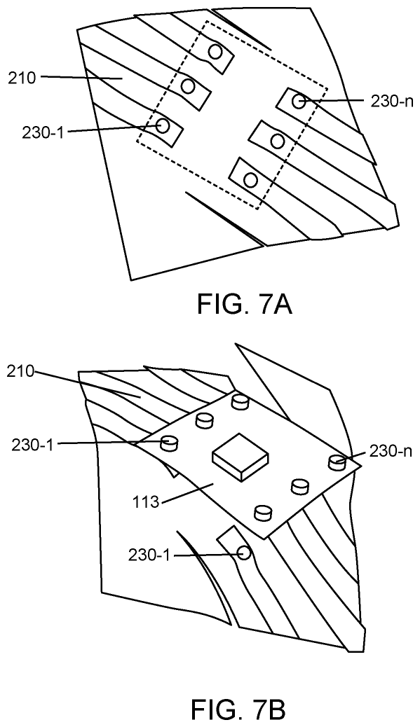

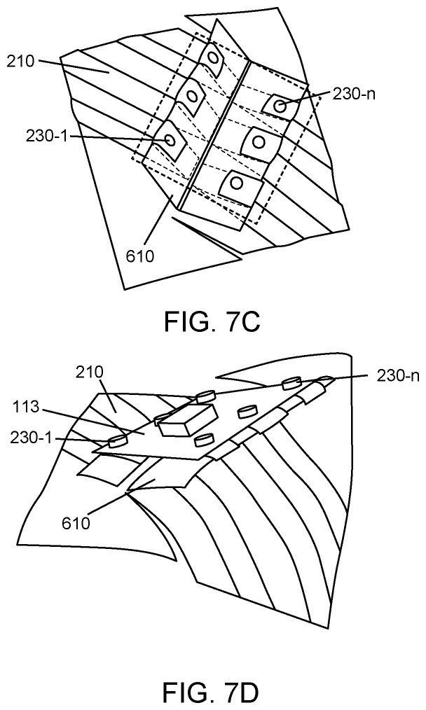

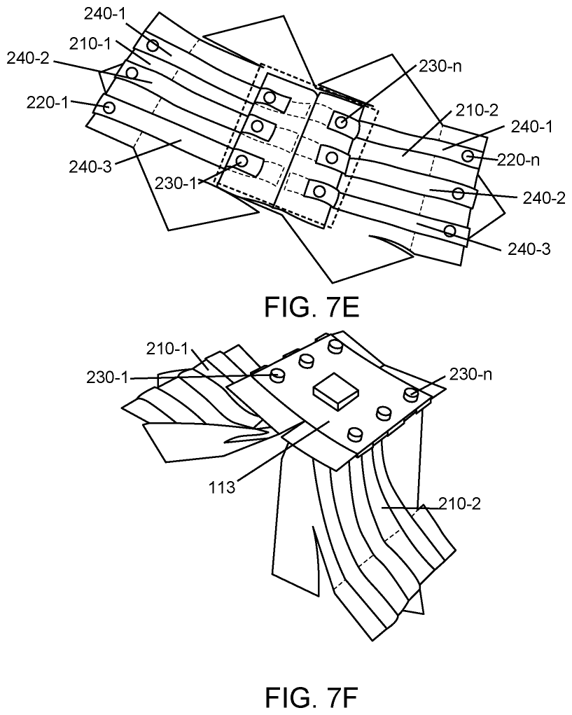

In FIG. 6B, sensor module 113 is depicted as being affixed to a wire module 210 comprising a flap structure 610. Sensor module 113 is affixed to a wire module 210 via functional module couplers 230-1 through 230-n (depicted in an exploded view). The flap structure 610 of wire module 210 will be discussed in greater detail herein with reference to FIGS. 7C-7D.

FIGS. 7A-7D depict example embodiments to connect a functional module to a single wire module 210; and FIGS. 7E-7F depict example embodiments to connect a functional module between two wire modules 210, in accordance with certain examples. FIG. 7A depicts a wire module 210 comprising functional module couplers 230-1 through 230-n. FIG. 7B depicts a functional module with functional module couplers 230-1 through 230-n in alignment with functional module coupler 230-1 of wire module 210 (functional module couplers 230-2 through 230-n for wire module 210 are not depicted). In an example, the functional module is sensor module 113, however, any functional module may be used.

FIG. 7C depicts a wire module 210 comprising functional module couplers 230-1 through 230-n and flap structure 610. Flap structure 610 is comprised of substrate 116. In an example, flap structure 610 has a width dimension that is half the width of the functional module. Flap structure 610 enables modular wearable device 110 to bend or flex without disengaging the functional modules. Flap structure 610 functions as a movable joint preventing the functional module from disconnecting. FIG. 7D depicts a functional module with functional module couplers 230 in alignment with functional module couplers 230-1 through 230-n across flap structure 610 of wire module 210. In an example, the functional module is sensor module 113, however, any functional module may be used.

FIG. 7E depicts wire modules 210-1 and 210-2 comprising wire module couplers 220-1 through 220-n and functional module couplers 230-1 through 230-n with conductors 240-1 through 240-3 in alignment. FIG. 7F depicts a functional module with functional module couplers 230-1 through 230-n in alignment with functional module couplers 230 (not depicted) of wire modules 210-1 and 210-2. In an example, the functional module is sensor module 113, however, any functional module may be used.

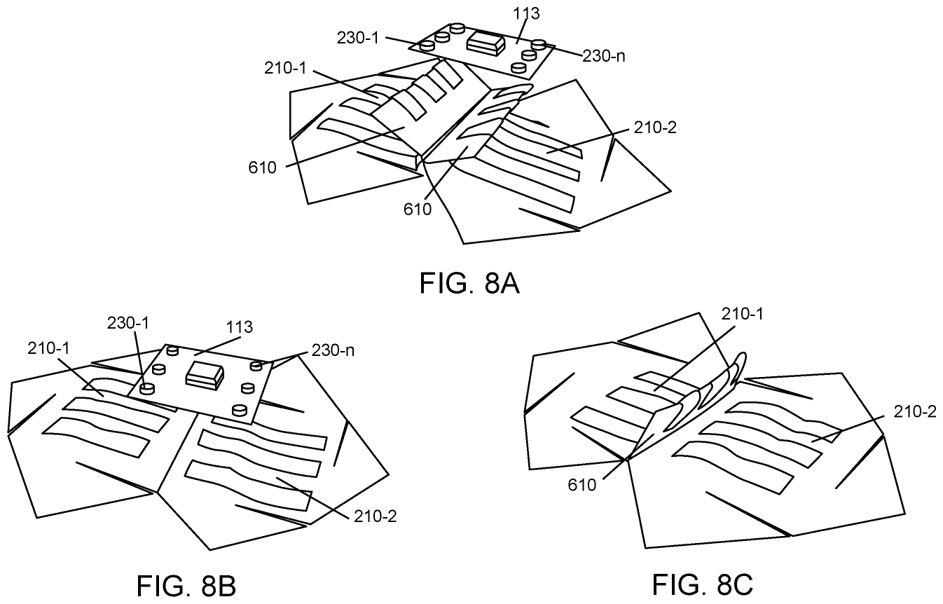

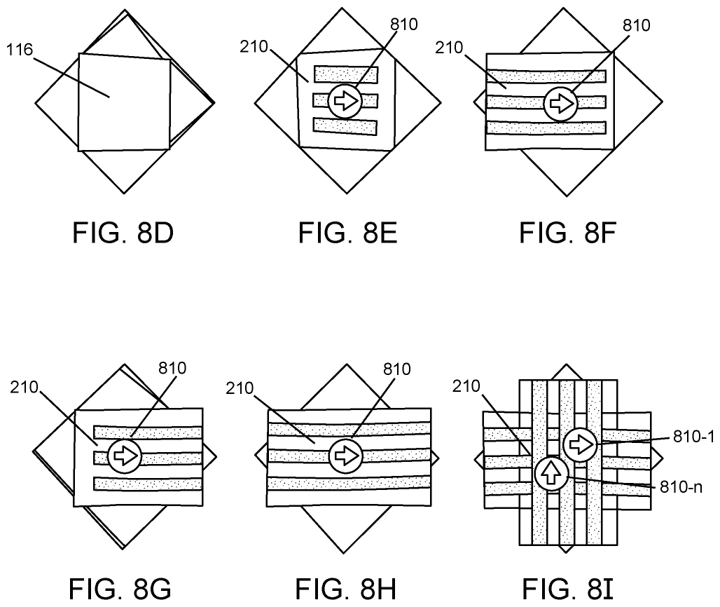

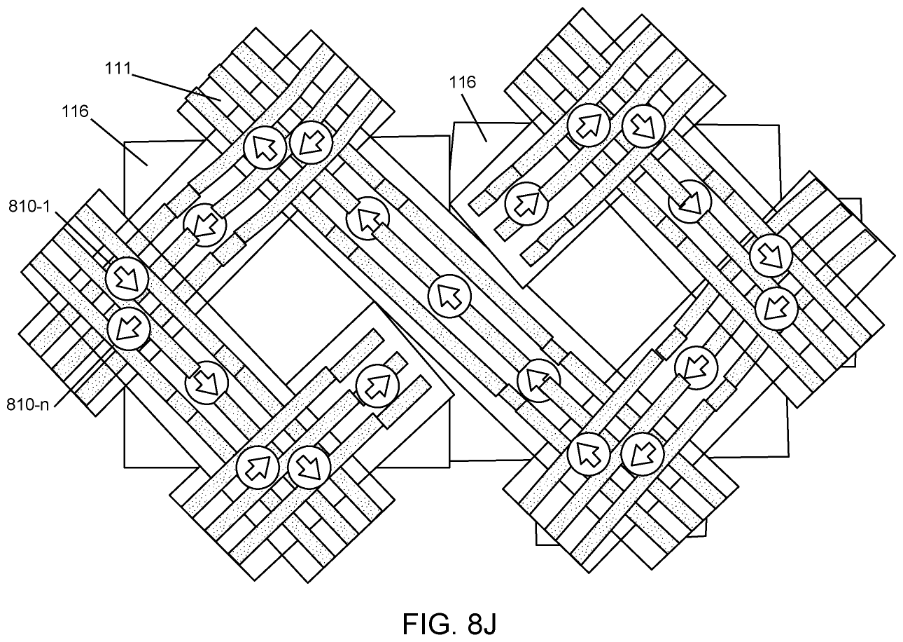

FIGS. 8A-8C depict example functional module connection configurations, FIGS. 8D-FIG. 8I depict example substrate 116 and wire module 210 configurations, and FIG. 8J depicts an example circuit layout of wire module assembly 111, in accordance with certain examples.

FIG. 8A depicts a flap-to-flap connection for a functional module, in an example sensor module 113, to be affixed to wire modules 210-1 and 210-2 via functional module connectors 230-1 through 230-n, each wire module 210 comprising a flap structure 610. FIG. 8B depicts a flat-to-flat connection for a functional module to be affixed to wire modules 210-1 and 210-2 via functional module connectors 230-1 through 230-n. FIG. 8C depicts a flap-to-flat connection for wire module 210-1 to be affixed to wire module 210-2 via wire module connectors 220 (not depicted).

FIG. 8D depicts a blank section of substrate 116, which may be used as a decorative module in modular wearable device. 110. FIG. 8E depicts a wire module 210 in a flat configuration. FIG. 8F depicts a wire module 210 in a flap-in configuration. FIG. 8G depicts a wire module 210 in a flap-out configuration. FIG. 8H depicts a wire module 210 in a double flap configuration. FIG. 8I depicts a wire module 210 in a cross-shape configuration. The one or more arrows 810 of FIGS. 8E-8I indicate the signal direction relative to conductors 240-1, 240-2, and 240-3. FIG. 8J depicts an example circuit layout of wire module assembly 111. FIG. 8J depicts a plurality of arrows 810-1 through 810-n indicating the direction of the signal relative to conductors 240-1, 240-2, and 240-3.

In an example, modular wearable device 110 may be configured to be affixed to a user. Modular wearable device 110 may comprise an adhesive layer such that modular wearable device 110 may be affixed to a location on a user. In an example, the adhesive layer may be a polyvinyl alcohol adhesive, an eyelash glue, a medical prosthetic adhesive, a nail adhesive, or any other suitable adhesive to affix modular wearable device 110 to a user. Modular wearable device 110 may be affixed to the skin of the user, the hair of the user, a garment of the user, or any suitable location such that modular wearable device 110 may detect one or more inputs from the environment in which wearable device 110 is located and provide a response to the one or more inputs.

### Other Examples

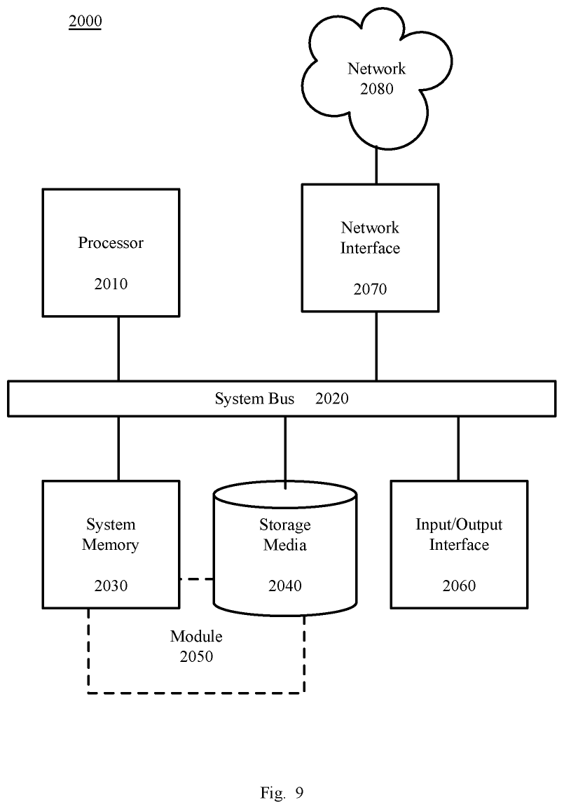

FIG. 9 depicts a computing machine 2000 and a module 2050 in accordance with certain examples. The computing machine 2000 may correspond to any of the various computers, servers, mobile devices, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a router or other network node, a vehicular information system, one or more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2600. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. The processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 0630 may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include WANs, LANs, intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. Any of the processor 2010, the other elements of the computing machine 2000, or the various peripherals discussed herein may be integrated into a single device such as a system on chip (“SOC”), system on package (“SOP”), or ASIC device.

Examples may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing examples in computer programming, and the examples should not be construed as limited to any one set of computer program instructions. Further, a skilled programmer would be able to write such a computer program to implement an example of the disclosed examples based on the appended flow charts and associated description in the application text. Therefore, disclosure of a particular set of program code instructions is not considered necessary for an adequate understanding of how to make and use examples. Further, those skilled in the art will appreciate that one or more aspects of examples described herein may be performed by hardware, software, or a combination thereof, as may be embodied in one or more computing systems. Moreover, any reference to an act being performed by a computer should not be construed as being performed by a single computer as more than one computer may perform the act.

The examples described herein can be used with computer hardware and software that perform the methods and processing functions described herein. The systems, methods, and procedures described herein can be embodied in a programmable computer, computer-executable software, or digital circuitry. The software can be stored on computer-readable media. Computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (“FPGA”), etc.

The systems, methods, and acts described in the examples presented previously are illustrative, and, alternatively, certain acts can be performed in a different order, in parallel with one another, omitted entirely, and/or combined between different examples, and/or certain additional acts can be performed, without departing from the scope and spirit of various examples. Accordingly, such alternative examples are included in the scope of the following claims, which are to be accorded the broadest interpretation so as to encompass such alternate examples.

Although specific examples have been described above in detail, the description is merely for purposes of illustration. It should be appreciated, therefore, that many aspects described above are not intended as essential elements unless explicitly stated otherwise. Modifications of, and equivalent components or acts corresponding to, the disclosed aspects of the examples, in addition to those described above, can be made by a person of ordinary skill in the art, having the benefit of the present disclosure, without departing from the spirit and scope of examples defined in the following claims, the scope of which is to be accorded the broadest interpretation so as to encompass such modifications and equivalent structures.

Various embodiments are described herein. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment,” “an embodiment,” “an example embodiment,” or other similar language means that a particular feature, structure, or characteristic described in connection with the embodiment is included in at least one embodiment of the invention described herein. Thus, appearances of the phrases “in one embodiment,” “in an embodiment,” “an example embodiment,” or other similar language in various places throughout this specification are not necessarily all referring to the same embodiment. Furthermore, the particular features, structures, or characteristics may be combined in any suitable manner in one or more embodiments, as would be apparent to a person having ordinary skill in the art and the benefit of this disclosure. Furthermore, while some embodiments described herein include some, but not other, features included in other embodiments, combinations of features of different embodiments are meant to be within the scope of the invention. For example, in the appended claims, any of the claimed embodiments can be used in any combination.

Example 1 is a wearable device, comprising: a plurality of conductive modules that when mutually affixed by one or more coupling devices form a circuit; a power module configured to be affixed to one or more of the plurality of conductive modules; at least one sensor module configured to be affixed to one or more of the plurality of conductive modules; and at least one actuator module configured to be affixed to one or more of the plurality of conductive modules, wherein the circuit is configured to receive an input from the at least one sensor module affixed to one or more of the plurality of conductive modules and to output a signal to the at least one actuator module affixed to one or more of the plurality of conductive modules to initiate a response function.

Example 2 includes the subject matter of Example 1, where the plurality of conductive modules comprises a minimum number of conductive modules suitable to encompass a location on a user where the response function is to be initiated.

Example 3 includes the subject matter of any of Examples 1-2, the power module configured to be interchangeable with a second power module.

Example 4 includes the subject matter of any of Examples 1-3, each of the at least one sensor modules configured to be interchangeable with one or more second sensor modules.

Example 5 includes the subject matter of any of Examples 1-4, each of the at least one actuator modules configured to be interchangeable with one or more second actuator modules.

Example 6 includes the subject matter of any of Examples 1-5, the one or more coupling devices comprising one or more of a fastener, a snap, a hook and loop, a magnet, or slits for tessellation.

Example 7 includes the subject matter of any of Examples 1-6, the one or more coupling devices comprising: one or more first coupling devices affixed to a first side of one of the conductive modules; and one or more second coupling devices affixed to a second side of another one of the conductive modules.

Example 8 includes the subject matter of any of Examples 1-7, the one or more first coupling devices configured to couple with the one or more second coupling devices such that each conductive module of the plurality of conductive modules is affixable to each of the other conductive modules of the plurality of conductive modules while maintaining conductivity to form the circuit.

Example 9 includes the subject matter of any of Examples 1-8, each conductive module of the plurality of conductive modules comprising: a conformable substrate; a first conductor affixed to the flexible substrate to provide power to the circuit; a second conductor affixed to the flexible substrate to provide ground to the circuit; and a third conductor affixed to the flexible substrate to transmit the signal from the at least one interchangeable sensor module to the at least one interchangeable actuator module.

Example 10 includes the subject matter of any of Examples 1-9, the conformable substrate comprising: a first silicone material layer; a first adhesive layer; a textile stabilizer material layer; a second adhesive layer; and a second silicone material layer, wherein the first silicone material layer is affixed to the textile stabilizer material layer by the first adhesive layer and the textile stabilizer material layer is affixed to the second silicone material layer by the second adhesive layer.

Example 11 includes the subject matter of any of Examples 1-10, the first conductor, the second conductor, and the third conductor comprising a flexible conductive material.

Example 12 includes the subject matter of any of Examples 1-11, the at least one sensor module comprising: a flexible printed circuit board; a sensor affixed to the flexible printed circuit board; a first microcontroller affixed to the flexible printed circuit board; and one or more third coupling devices configured to couple with the one or more coupling devices such that the at least one sensor module is affixed to one or more of the plurality of conductive modules.

Example 13 includes the subject matter of any of Examples 1-12, the first microcontroller configured to: receive the input from the sensor; and output the signal to the at least one actuator module to initiate the response function.

Example 14 includes the subject matter of any of Examples 1-13, wherein the sensor comprises a capacitive touch sensor.

Example 15 includes the subject matter of any of Examples 1-14, wherein the sensor comprises a resistive sensor.

Example 16 includes the subject matter of any of Examples 1-15, wherein the sensor comprises a strain sensor.

Example 17 includes the subject matter of any of Examples 1-16, wherein the sensor comprises a pressure sensor.

Example 18 includes the subject matter of any of Examples 1-17, wherein the sensor comprises a biosensor.

Example 19 includes the subject matter of any of Examples 1-18, the biosensor configured to detect one or more of a temperature, blood pressure, or pulse of a user.

Example 20 includes the subject matter of any of Examples 1-19, wherein the sensor comprises a light sensor.

Example 21 includes the subject matter of any of Examples 1-20, wherein the sensor comprises an ultraviolet (“UV”) light sensor.

Example 22 includes the subject matter of any of Examples 1-21, wherein the sensor comprises an environmental gas sensor.

Example 23 includes the subject matter of any of Examples 1-22, wherein the sensor comprises a proximity sensor.

Example 24 includes the subject matter of any of Examples 1-23, wherein the sensor is extendable relative to the flexible printed circuit board.

Example 25 includes the subject matter of any of Examples 1-24, the at least one actuator module comprising: a flexible printed circuit board; an actuator affixed to the flexible printed circuit board; a second microcontroller affixed to the flexible printed circuit board; and one or more fourth coupling devices configured to couple with the one or more coupling devices such that the at least one actuator module is affixed to one or more of the plurality of conductive modules.

Example 26 includes the subject matter of any of Examples 1-25, the second microcontroller configured to: receive the signal from the at least one sensor module; and output a second signal to the actuator to initiate the response function.

Example 27 includes the subject matter of any of Examples 1-26, wherein the actuator comprises a haptic feedback component.

Example 28 includes the subject matter of any of Examples 1-27, wherein the haptic feedback component comprises a shape-memory alloy actuator configured to apply a force, a vibration, a thermal sensation, or a motion as the response function.

Example 29 includes the subject matter of any of Examples 1-28, wherein the actuator comprises a stiffness component.

Example 30 includes the subject matter of any of Examples 1-29, the stiffness component comprising a shape memory alloy configured to enable variable-stiffness as the response function.

Example 31 includes the subject matter of any of Examples 1-30, wherein the actuator comprises a thermochromic display.

Example 32 includes the subject matter of any of Examples 1-31, wherein the actuator comprises an audio device.

Example 33 includes the subject matter of any of Examples 1-32, wherein the actuator comprises a light emitting diode (“LED”).

Example 34 includes the subject matter of any of Examples 1-33, wherein the actuator comprises a light emitting diode (“LED”) array.

Example 35 includes the subject matter of any of Examples 1-34, wherein the actuator comprises an organic light emitting diode (“OLED”).

Example 36 includes the subject matter of any of Examples 1-35, wherein the actuator comprises a photochromic display.

Example 37 includes the subject matter of any of Examples 1-36, wherein the actuator comprises a buzzer.

Example 38 includes the subject matter of any of Examples 1-37, wherein the actuator is extendable relative to the flexible printed circuit board.

Example 39 includes the subject matter of any of Examples 1-38, the power module comprising one or more batteries to provide power to the wearable device.

Example 40 includes the subject matter of any of Examples 1-39, the one or more batteries comprising one or more of a lithium polymer battery, a lithium ceramic battery, or a triboelectric nanogenerator.

Example 41 includes the subject matter of any of Examples 1-40, further comprising at least one modifier module configured to be affixed to one or more of the plurality of conductive modules.

Example 42 includes the subject matter of any of Examples 1-41, the at least one modifier module comprising: a flexible printed circuit board; a modifying device affixed to the flexible printed circuit board; a third microcontroller affixed to the flexible printed circuit board; and one or more fifth coupling devices configured to couple with the one or more coupling devices such that the at least one modifier module is affixed to one or more of the plurality of conductive modules.

Example 43 includes the subject matter of any of Examples 1-42, the third microcontroller configured to: receive the signal from the at least one sensor module; transform the signal to initiate an altered response function; and output the transformed signal to the at least one actuator module to initiate the altered response function.

Example 44 includes the subject matter of any of Examples 1-43, wherein the modifying device comprises an inverter.

Example 45 includes the subject matter of any of Examples 1-44, wherein the modifying device comprises a signal modifier, the signal modifier configured to alter an amplitude associated with a response function comprising light, volume, vibration, or heat.

Example 46 is a method, comprising: by a processor of a wearable device, the wearable device comprising a plurality of conductive modules mutually affixed to form a circuit, one or more sensor modules, and one or more actuator modules: receiving an input from the one or more sensor modules of the wearable device; based on the input, determining a response function to be performed by the one or more actuator modules of the wearable device; and transmitting, to each of the one or more actuator modules via the circuit, instructions to perform the responsive function.

Example 47 is a method to construct a wearable device, comprising: selecting a set of conductive modules; affixing each conductive module of the set of conductive modules to one or more conductive modules of the set of conductive modules to form a circuit to enable a response function of a wearable device; affixing a power module to at least one conductive module of the set of conductive modules; affixing at least one sensor module to at least one conductive module of the set of conductive modules; and affixing at least one actuator module to at least one conductive module of the set of conductive modules, wherein the circuit is configured to receive an input from the at least one sensor module and to output a signal to the at least one actuator module to initiate the response function.

Example 48 includes the subject matter of Example 47, wherein selecting the set of conductive modules comprises determining a minimum number of conductive modules suitable to encompass a location on a user where the response function is to be initiated.

Example 49 includes the subject matter of any of Examples 47-48, wherein the response function comprises one or more of a force, a vibration, a thermal sensation, a motion, a change in stiffness, a light, or an audible sound.

Example 50 includes the subject matter of any of Examples 47-49, further comprising: removing the at least one sensor module from the at least one conductive module of the set of conductive modules; affixing at least one second sensor module to at least one conductive module of the set of conductive modules; removing the at least one actuator module from the at least one conductive module of the set of conductive modules; affixing at least one second actuator module to at least one conductive module of the set of conductive modules, wherein the circuit is configured to receive an input from the at least one second sensor module and to output a signal to the at least one second actuator module to initiate a second response function.

1. A wearable device, comprising:
a plurality of conductive modules that when mutually affixed by one or more coupling devices form a circuit;
a power module configured to be affixed to one or more of the plurality of conductive modules;
at least one sensor module configured to be affixed to one or more of the plurality of conductive modules; and
at least one actuator module configured to be affixed to one or more of the plurality of conductive modules,
wherein the circuit is configured to receive an input from the at least one sensor module affixed to one or more of the plurality of conductive modules and to output a signal to the at least one actuator module affixed to one or more of the plurality of conductive modules to initiate a response function.

2. The wearable device of claim 1, wherein the plurality of conductive modules, is configured to connect two or more functional modules selected from a power module, a sensor module, and an actuator module, and, optionally, a modifier module; and wherein each of the conductive modules and each of the functional modules are configured to be interchangeable, reusable, reconfigurable, detachable, and re-attachable.

3. (canceled)

4. (canceled)

5. (canceled)

6. (canceled)

7. The wearable device of claim 1, wherein the two or more coupling devices comprise:
one or more first coupling devices affixed to a first side of one of the conductive modules; and
one or more second coupling devices affixed to a second side of another one of the conductive modules; and
wherein the two or more coupling devices comprising one or more of a fastener, a snap, a hook and loop, a magnet, or slits for tessellation.

8. (canceled)

9. The wearable device of claim 1, each conductive module of the plurality of conductive modules comprises:
a conformable substrate;
a first conductor affixed to the flexible substrate to provide power to the circuit;
a second conductor affixed to the flexible substrate to provide ground to the circuit; and
a third conductor affixed to the flexible substrate to transmit the signal from the at least one interchangeable sensor module to the at least one interchangeable actuator module.

10. The wearable device of claim 9, the conformable substrate comprising:
a first silicone material layer;
a first adhesive layer;
a textile stabilizer material layer;
a second adhesive layer; and
a second silicone material layer, wherein the first silicone material layer is affixed to the textile stabilizer material layer by the first adhesive layer and the textile stabilizer material layer is affixed to the second silicone material layer by the second adhesive layer.

11. The wearable device of claim 9, the first conductor, the second conductor, and the third conductor comprise a flexible conductive material.

12. The wearable device of claim 1, the at least one sensor module comprises:
a flexible printed circuit board;
a sensor affixed to the flexible printed circuit board;
a first microcontroller affixed to the flexible printed circuit board; and
one or more third coupling devices configured to couple with the one or more coupling devices such that the at least one sensor module is affixed to one or more of the plurality of conductive modules.

13. The wearable device of claim 12, the first microcontroller configured to:
receive the input from the sensor; and
output the signal to the at least one actuator module to initiate the response function.

14. The wearable device of claim 12, wherein the sensor comprises a capacitive touch sensor, a resistive sensor, a strain sensor, a pressure sensor, a biosensor, a light sensor, an ultraviolet (“UV”) light sensor, a proximity sensor, or an environmental gas sensor; and wherein the sensor is extendable relative to the flexible printed circuit board.

15. (canceled)

16. (canceled)

17. (canceled)

18. (canceled)

19. (canceled)

20. (canceled)

21. (canceled)

22. (canceled)

23. (canceled)

24. (canceled)

25. The wearable device of claim 1, the at least one actuator module comprises:
a flexible printed circuit board;
an actuator affixed to the flexible printed circuit board, wherein the actuator is extendable
relative to the flexible printed circuit board;
a second microcontroller affixed to the flexible printed circuit board; and
one or more fourth coupling devices configured to couple with the one or more coupling devices such that the at least one actuator module is affixed to one or more of the plurality of conductive modules.

26. The wearable device of claim 25, wherein the second microcontroller is configured to:
receive the signal from the at least one sensor module; and
output a second signal to the actuator to initiate the response function.

27. The wearable device of claim 25, wherein the actuator comprises a haptic feedback component, a stiffness component, a thermochromic display, an audio device, a light emitting diode (“LED”), a light emitting diode (“LED”) array, an organic light emitting diode (“OLED”), a photochromic display, a buzzer, or any combinations thereof.

28. The wearable device of claim 25, wherein the actuator comprises a haptic feedback component and the haptic feedback component comprises a shape-memory alloy actuator configured to apply a force, a vibration, a thermal sensation, or a motion as the response function.

29. (canceled)

30. The wearable system of claim 25, wherein the actuator comprises a stiffness component and the stiffness component comprising a shape memory alloy configured to enable variable-stiffness as the response function.

31. (canceled)

32. (canceled)

33. (canceled)

34. (canceled)

35. (canceled)

36. (canceled)

37. (canceled)

38. (canceled)

39. The wearable device of claim 1, the power module comprising one or more batteries to provide power to the wearable device; and wherein the one or more batteries comprising one or more of a lithium polymer battery, a lithium ceramic battery, or a triboelectric nanogenerator.

40. (canceled)

41. The wearable device of claim 1, further comprising at least one modifier module configured to be affixed to one or more of the plurality of conductive modules, wherein the at least one modifier module comprising:
a flexible printed circuit board;
a modifying device affixed to the flexible printed circuit board;
a third microcontroller affixed to the flexible printed circuit board; and
one or more fifth coupling devices configured to couple with the one or more coupling
devices such that the at least one modifier module is affixed to one or more of the plurality
of conductive modules.

42. (canceled)

43. The wearable device of claim 41, the third microcontroller configured to:
receive the signal from the at least one sensor module;
transform the signal to initiate an altered response function; and
output the transformed signal to the at least one actuator module to initiate the altered response function.

44. The wearable device of claim 42, wherein the modifying device comprises an inverter and a signal modifier, wherein the signal modifier alters an amplitude associated with a response function comprising light, volume, vibration, or heat.

45. (canceled)

46. The wearable device of claim 2, wherein at least a portion of the plurality of conductive modules are substantially identical in size and shape; and wherein the two or more functional modules are substantially identical in size and shape.

47. The wearable device of claim 2, wherein each of the plurality of conductive modules, and/or each of the two or more functional modules further comprises a multi-layer conformable substrate conforms and adheres to skin.
